(19) United States
(12) Patent Application Publication
(10) Pub. No.: US 2008/0097431 A1
(43) Pub. Date: Apr. 24, 2008

(54) Flexible spinal stabilization

(76) Inventor: Paul Peter Vessa, Bernardsville, NJ (US)

Correspondence Address: KENYON & KENYON LLP, 1500 K STREET N.W., SUITE 700, WASHINGTON, DC, 20005, US

(21) Appl. No.: 11/525,050

(22) Filed: Sep. 22, 2006

(51) Int. Cl. A61B 17/58; A61B 17/56

(52) U.S. Cl. 606/61; 606/73

(57) ABSTRACT

The invention regards spinal stabilization. It may include a spinal support system having spinal anchors and a bridge coupled to the anchors wherein the bridge has a length with a more flexible section and a less-flexible section. The less flexible section may be at an end of the bridge and the more flexible section may be off centered between the two spinal anchors. It may also include a kit having some or all of these components as well as spacers. It may further include a method of designing a spinal stabilization system this method may include identifying three-dimensional loads placed at a location of a spinal column, identifying three-dimensional ranges of motion for that location of the spinal column, quantifying the forces associated with the identified loads, and designing a spring bridge to transfer some but not all of the loads for at least one of the axes from one end of the spring bridge to another end of the spring bridge, the load not transferred being absorbed at least partially by flexure of the spring bridge.

### FIELD OF THE INVENTION

The present invention regards providing flexible supports for a spinal column. More specifically, the present invention regards a flexible connection system for linking vertebrae of a spinal column, kits containing these flexible systems, and methods for designing and installing these flexible systems.

### BACKGROUND OF THE INVENTION

The human spinal column consists of a series of thirty-three stacked vertebrae. Each vertebrae is separated by a disc and includes a vertebral body having several posterior facing structures. These posterior structures include pedicles, lamina, articular processes, and spinous process. The articular processes, which function as pivoting points between vertebrae, include left and right superior and inferior processes. The superior and inferior processes of adjacent vertebrae mate with each other to form joints called facet joints. In a typical pair of vertebrae, the inferior articular facet of an upper vertebrae mates with the superior articular facet of the vertebra below to form a facet joint.

The facet joints of the spinal column contribute to the movement and the support of the spine. This movement and rotation is greatest in the cervical (upper) spine region and more restrictive near the lumbar (lower) spine region. In the cervical region of the spine, the articular facets are angled and permit considerable flexion, extension, lateral flexion, and rotation. In the thoracic region, the articular facets are oriented in the coronal plane and permit some rotation, but no flexion or extension. In the lumbar region of the spinal column, the articular facets are oriented in a parasagittal plane and permit flexion, extension, and lateral bending but they limit rotation.

Through disease or injury, the posterior elements of the spine, including the facet joints of one or more vertebrae, can become damaged such that the vertebrae no longer articulate or properly align with each other. This can result in a misaligned anatomy, immobility, and pain. As such, it is sometimes necessary to remove part or all of the facet joint with a partial or full facetectomy. Removal of facet joints, however, destabilizes the spinal column as adjacent stacked vertebrae can no longer fully interact with and support each other.

One way to stabilize the spinal column after removal of facet joints or other posterior elements of the spine is to vertically rigidly fix adjacent stacked vertebrae through bone grafting and/or rigid mechanical fixation assemblies. In each case, the adjacent vertebrae are rigidly fixed to one another through a medical procedure. In these fixed systems, the spine looses flexibility as two previously moveable vertebrae are fused a certain distance apart from one another and, consequently, function and move as a single unit.

### SUMMARY OF THE INVENTION

Embodiments of the present invention may be used to link or otherwise connect vertebrae of the spine. These connections may be made with screws or other anchors fixedly connected to the vertebrae and a bridge linking the embedded anchors together. In embodiments of the invention, the bridge and / or the anchors may be configured to mimic the natural connections of vertebrae of the spine. This may include sizing the dimensions of the bridge such that it opposes the physical forces placed on it much in the same manner as the natural connections the bridge is replacing or supplementing. In some instances, the bridge and anchors may be configured to reduce or absorb the amount of force exerted on the anchors. When these forces are reduced, the likelihood that the anchor will be rocked loose of the vertebrae in which it is seated may be reduced.

Over its lifetime, a spinal support system may experience cyclical loading that exceeds millions of cycles. In each cycle of loading an anchor may experience a pushing load and a pulling load, in other words a tension load and a compression load. These loads may contain force vector components that directly oppose each other. These opposing forces, which result in the repeated loading and unloading of an anchor over its lifetime can work to loosen and or decay the connection between the anchor and the vertebrae. This decay can result as small bone fissures and cracks are created near the bone anchor interface due to the rocking motion or opposing forces. Overtime this can cause decreased performance and even failure of an anchor system. Comparatively, in embodiments of the present invention the forces placed on the anchors may be reduced or more efficiently distributed to the anchors. Through such designs and installations, embodiments of the present invention, once installed by a practitioner, may remain in-situ for prolonged periods of time.

Embodiments of the present include support systems that can have two spinal anchors and a bridge linking them. In some embodiments, this bridge may be designed and configured to absorb energy and not to directly transfer energy from one anchor to the other. In so doing, the forces placed on the anchors may be reduced. In some embodiments the bridge may be a flat spring having a coiled section and a solid section. The turns of the spring in the coiled section may be designed to have a rectangular cross-section and may be further designed such that the longer face may withstand higher shear forces than the more narrow section. Concurrently, the narrow section may be designed to allow the spring to flex when non-axial forces are exerted on the spring. This flexure can act to absorb energy and to reduce the likelihood that the anchors will become dislodged from spinal bone in which they are anchored.

In some embodiments of the invention bridging springs having a variety of strength characteristics may be assembled into a kit. By combining the springs or other bridges in this fashion, a practitioner may select the bridge that most closely mimics the natural spinal supports that the bridge will be replacing or supplementing. A kit in accord with the invention may also include other components such as spacers and anchors, which are themselves configured to couple with the various bridges of the kit. In some instances, the bridge may contain a coiled portion and a solid portion, wherein the coiled portion is positioned off of the installed center of the bridge. These and other examples of the invention are described herein.

While various embodiments of the invention are provided, these are not the only plausible embodiments. For instance, components of the various systems may be switched between embodiments and added or deleted from the embodiments. Likewise, the methods described herein may be performed in various sequences and may include fewer and more steps without departing from the teaching of the present invention.

### BRIEF DESCRIPTION OF THE DRAWINGS

FIG. 1 is an exploded view of a spinal support system in accord with the present invention.

FIG. 2 is a side view of an assembled spinal support system in accord with the present invention.

FIG. 3 is a top view of an assembled spinal support system in accord with the present invention.

FIG. 4 is an elevation of two spinal support systems installed in a spinal column in accord with the present invention.

FIG. 5 is a sectional view of a coiled section of a spring from a spinal support system in accord with the present invention.

FIG. 6 is a sectional view of a coiled section of the spring of the spinal support system in a regular, compressed, and expanded position.

FIG. 7a is a side view of a spring for a spinal support system in accord with the present invention.

FIG. 7b is an end view of the spring from FIG. 7a.

FIG. 7c is a sectional view along line A-A of FIG. 7a.

FIG. 7d is an isometric view of a spring for a spinal support system of the present invention.

FIG. 8 is a side view of a spring and two spacers as may be employed in the current invention.

FIG. 9 is a plan-view of a kit containing springs and spacers as may be employed in the current invention.

FIG. 10 is a rear-view of a spinal column having pedicle screws installed and inserts installed in the pedicle screws prior to the installation of a spring in accord with the present invention.

FIG. 11 is a rear view of the spinal column showing installed pedicle screws and a spring in accord with the present invention.

FIG. 12 is a flow chart explaining a method that may be employed in accord with the present invention.

### DETAILED DESCRIPTION

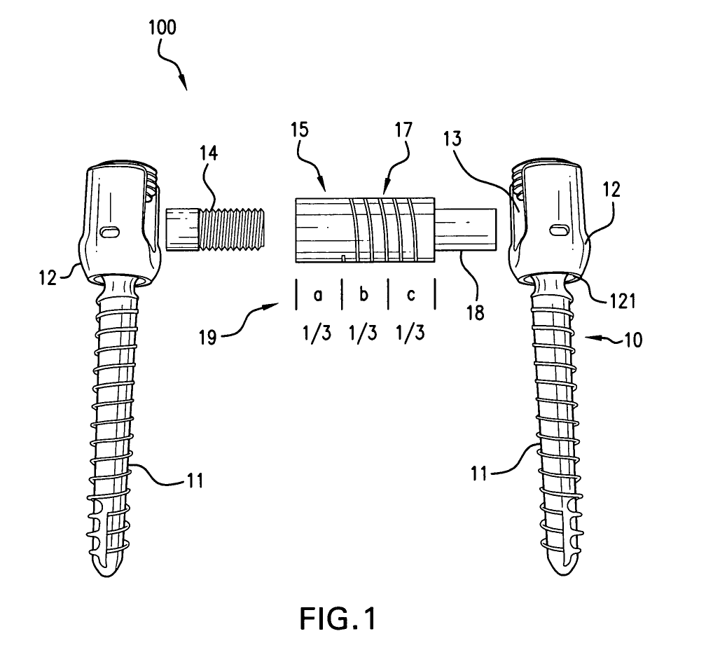

FIG. 1, which is an exploded view of a spinal support system as may be employed in accord with the current invention, shows two pedicle screws 10, each pedicle screw having a screw head 12 and screw threads 11. The screw head 12 is shown coupled to the screw threads 11 through a ball joint 121. FIG. 1 also shows an insert 14 and a bridging spring 16. As can be seen, the bridging spring 16 contains three portions: a solid section 15, a coiled section 17, and an insert section 18. Also shown in FIG. 1 are spring distance markers. As can be seen from these markers, approximately one-third of a larger diameter section of the spring 16 is a non-coiled or solid portion 15, while two-thirds of this larger diameter section of the spring 16 is a coiled section 17. As can also be seen in FIG. 1, the insert section 18 is roughly one-third of the overall length of the spring 16 and is also roughly equal to the combined length of the solid section 15 and the spring section 16. Insert 14 may be sized to slide within the solid section 15 and also to slide within the screw head 12, allowing it to be secured within the screw head 12. Likewise, the insert 18 is also sized to slide within the screw head 12. Area 13 of the screw head 12 is shown as the area in which the inserts may be secured.

In use, pedicle screw 10 may be installed into a pedicle of the spine that has been previously re-sectioned or otherwise is in need of repair. Once the screws 10 are installed, the spring may be positioned between the screw heads 12 and secured to the screw heads 12. By connecting the screw heads 12 with the spring 16 and insert 14, forces may be transferred between the screw heads 12, providing support to the spinal column in which the screw heads are anchored and mimicking the natural connections that have been replaced and/or are being supplemented by the spinal support system 100.

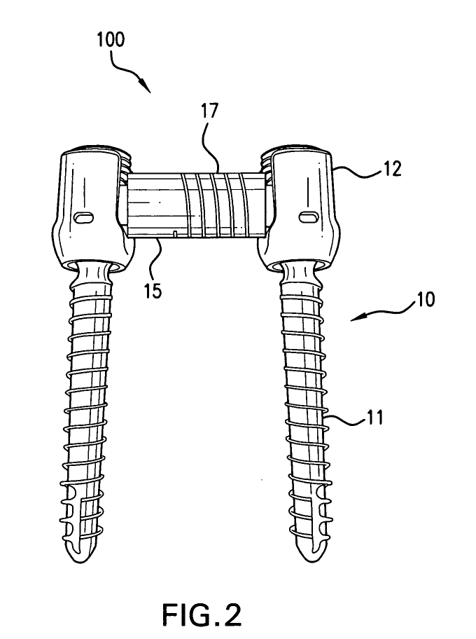

FIG. 2 shows the spinal support system 100 in a collapsed configuration as may occur when the system is installed. As can be seen in FIG. 2, once the inserts 18 and 14 are secured in the pedicle head 12, they are no longer visible as they may fit completely within the screw head 12 and within the solid section 15 of the spring 16. As can also be seen in FIG. 2, the coil section 17 is approximately two-thirds of the exposed portion of the spring 16 once the spring 16 is installed between the screw heads 12. By positioning the spring in this fashion the spinal support system may be more flexible on one side and less flexible on the other sides, the side with the solid portion 15. This differing flexibility may better mimic the support and movement provided previously by a facet joint or other connection that the spinal support system 100 replaces or supplements.

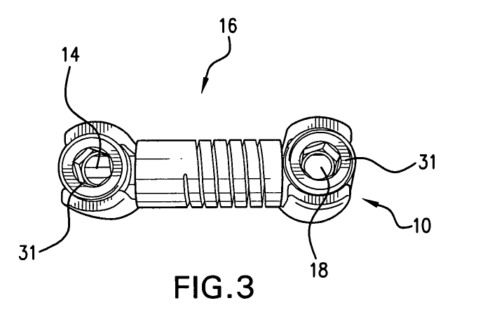

FIG. 3 is a top view of the spinal support system from the previous figures. Visible in this view are the insert 14 and the insert 18. As can be seen, each of these inserts may fit within the screw head 12 and may be secured to the screw head 12 by screws 31. By screwing down the screws 31 in the screw heads 12, the inserts 14 and 18 may be secured such that they are less likely to pull out of the pedicle screw heads 12 and also less likely to rotate within the pedicle screw heads 12. A groove or other indentation may be cut into the inserts to further reduce the likelihood of rotation and to help align the inserts.

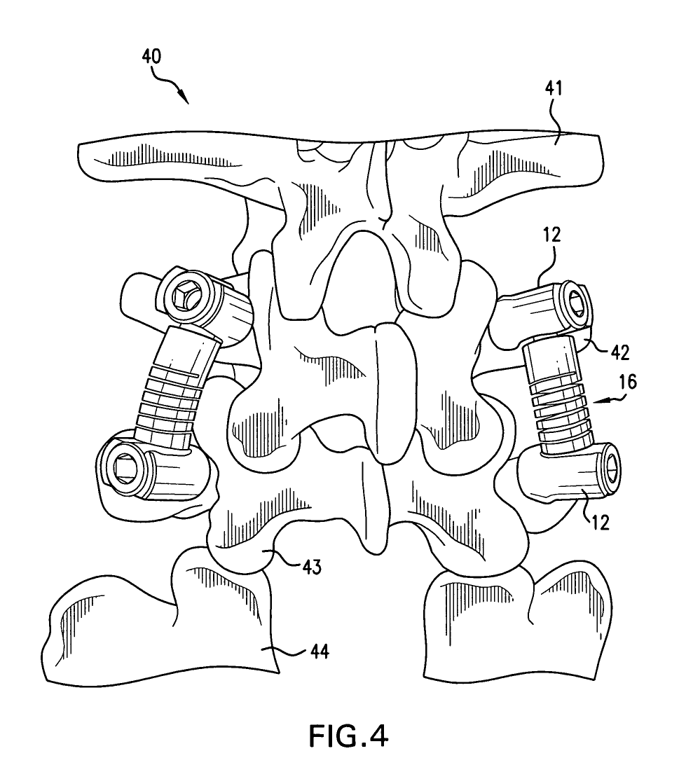

FIG. 4 is a rear view of a spinal column 40 with lumbar vertebrae 41, 42, 43, and 44, having two of the spinal support systems installed. As can be seen in this figure, the coiled section of the spring 16 is positioned below the solid portion 15 of the spring. This positioning may be used to better mimic the facet joints being replaced by the system 100. As can also be seen, the screws 31 are turned to be tight and nearly flush, if not completely flush, with the tops of the screw heads 12.

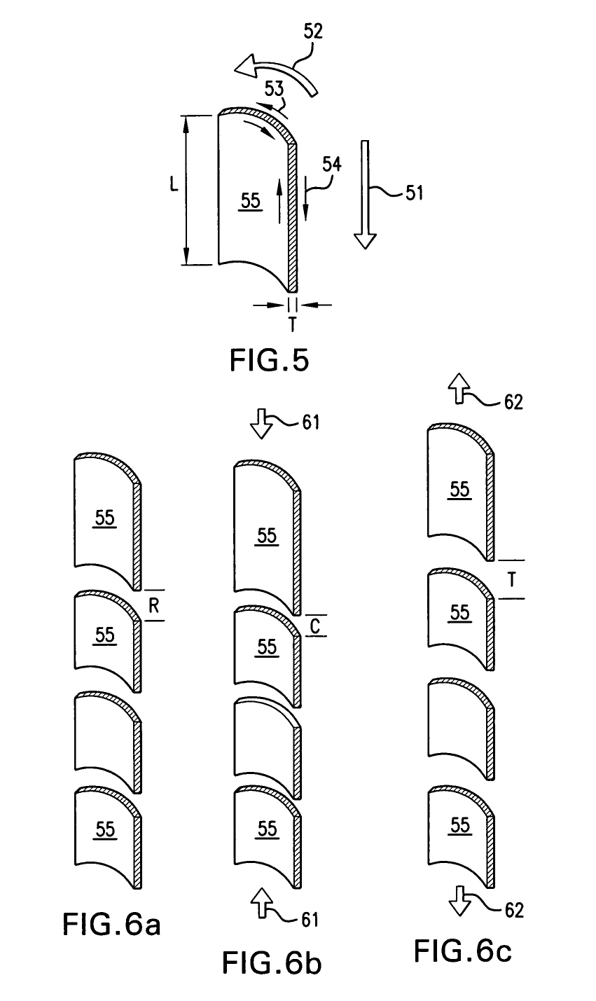

FIG. 5 is a sectional view of a spring section of a bridging spring of an embodiment of the present invention. FIG. 5 shows torsion forces 52 and longitudinal shear forces 31 being placed on the spring. As can be seen in FIG. 5, the coils of the spring have a rectangular cross-section with a length 1 and a thickness t. As described in more detail below, the length 1 and thickness t may be selected such that shear forces may be effectively transferred to the anchors while at the same time unwanted forces may not be transferred from the spring to the screw heads or these forces may be reduced or otherwise absorbed. One reason to reduce the amount of force transferred to the screw heads is to reduce the likelihood that the screw threads 11 anchored into the bone of the vertebrae will become loose over time by excessive loading on the anchor. By configuring the spring size and spacing in this fashion, the spinal support system may provide adequate support to the vertebrae while at the same time reducing the likelihood that the system will be overly rigid and transmit unnecessary forces to the anchors.

FIG. 6 shows three examples of a sectional view of a coiled portion of a bridging spring under different loading conditions in accord with the present invention. On the left, in column a, the spring sections 55 are spaced a regular distance apart. In the middle column, column b, the spring sections 55 are shown closer together because the spring is bearing a compressed load. In the right column, column c, the spring coils are under an expansive load such that the spacing E between each coil is larger than the spaces of the previous two loaded conditions. The ability of the coils of the spring to move in this fashion can serve to absorb energy and thereby reduce the load transferred between anchors.

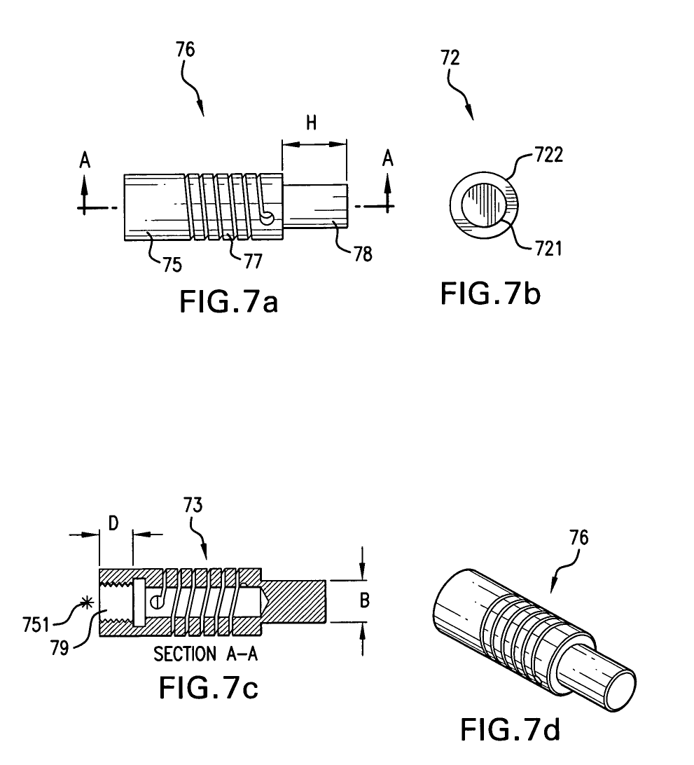

FIGS. 7a through 7d show side, sectional, and isometric views of a bridging spring in accord with the present invention. FIG. 7a is a side view of the bridging spring 76. This spring 7b contains a solid section 75, a coiled section 77, and an insert section 78. The solid section and the coiled section have a larger diameter than the insert section 78. The coiled section contains a bore hole indicating the end of the coils is the section. The bridging spring may be made from medical grade titanium as well as other materials such as nitinol. The spring may also have shape memory characteristics such that it reverts to a previous shape after the spring is installed and exposed to the heat of the body.

FIG. 7b is an end view of the spring 76. As can be seen in this view, the spring outer diameter 722 is greater and larger than the diameter of the insert 78. As can also be seen, the insert 78 has a circular cross section.

FIG. 7c is a sectional view taken along line A-A of FIG. 7a. FIG. 7c shows the bore 79, the center line 731 of the spring 76, and the inserts 78 width at B, also shown is the depth of bore D. Visible in this figure is the spacing between each of the coils of the coil section, as well as a cross-sectional view of each one of these coils.

FIG. 7d is an isometric view of the spring 76.

Springs that embody the invention may have various shapes and sizes. In the embodiment shown in FIGS. 71-7d, the spring may have a diameter of 10.0 mm, a bore depth of 5.0 mm, a insert length of 10.0 and an insert diameter of 6.5 mm. It may be made from titanium and may be designed to withstand 2.5 K/N in axial loading, 4.0 Nm/deg in torsional loading, 850 lb/in in lateral loading, and 2.0 Nm/deg of bending forces. Preferred performance characteristics of the spring include an axial deflection of 0.36 N/mm, a torsional deflection of 1.476 lb-in/deg lateral deflection of 91 N/mm and bending deflection of 0.96 Nm/deg.

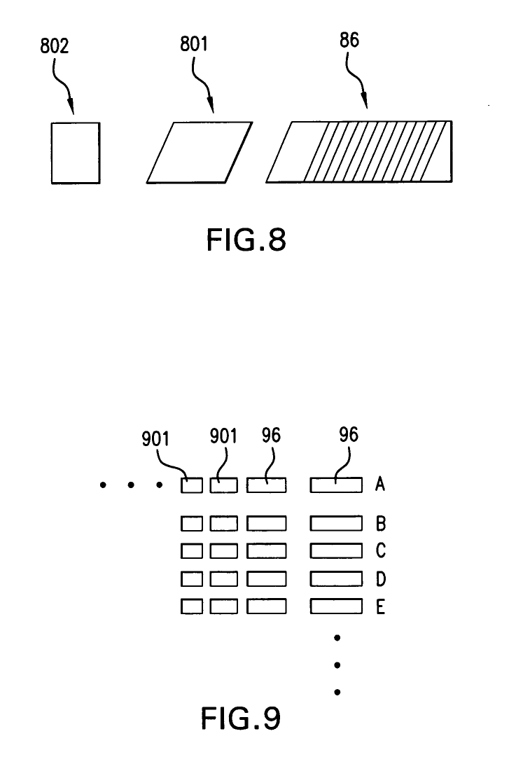

FIG. 8 is a side view of a spring and dual spacer configuration that may be employed in accord with the present invention. The spring 86 may be used in conjunction with a spacer 801 and a spacer 802 in order to provide adequate torsional resistance and/or to properly space the spring between installed pedicle screws 10. In other words, after pedicle screws are installed by a practitioner, should the spring 86 be unable to adequately bridge the gap between the screws, spacers 801 and 802 may be added in order to connect the installed pedicle screws. By using this hybrid configuration, different pedicle screw spacings may be accommodated.

FIG. 9 is a plan view of a kit that may be used in accord with the present invention. This kit may contain springs 96 and spacers 91. These springs and spaces may vary in length, strength, and in configuration. These springs may include any of the springs disclosed in this specification. They may be placed in rows A through E according to their design criteria. An advantage of such a kit is that prior to a practitioner installing the necessary spinal support, the practitioner may evaluate the patent contemporaneously with the procedure and may choose from the various springs and spacers to best suit the situation. Consequently, a practitioner may select a heavy spring with more rigid characteristics for the lumbar area and a lighter spring, that is more flexible for the thoracic area. The practitioner may also select one or more spacers to bridge the gap between installed anchors. These spacers may also have different strength and bending characteristics, so the practitioner may also choose them based on these characteristics as well. These selections may be made contemporaneous with the medical procedure as well as before. By offering the kit and selecting the spring contemporaneous with the procedure, a practitioner may evaluate the trauma and resectioned area and determine the best support system characteristics at that time. In other words, thepractitioner may see that while the third and fourth lumbar vertebrae are being connected, given the low weight of the patient a more bendable and lighter spring may be warranted than originally planned. This adjustment may be made by selecting a different spring and if necessary spacer system form the kit. Likewise, if the anchors are positioned further away than previously planned, additional spacers may be employed to position the flexible end of the spring and solid end of the spring properly between the anchors.

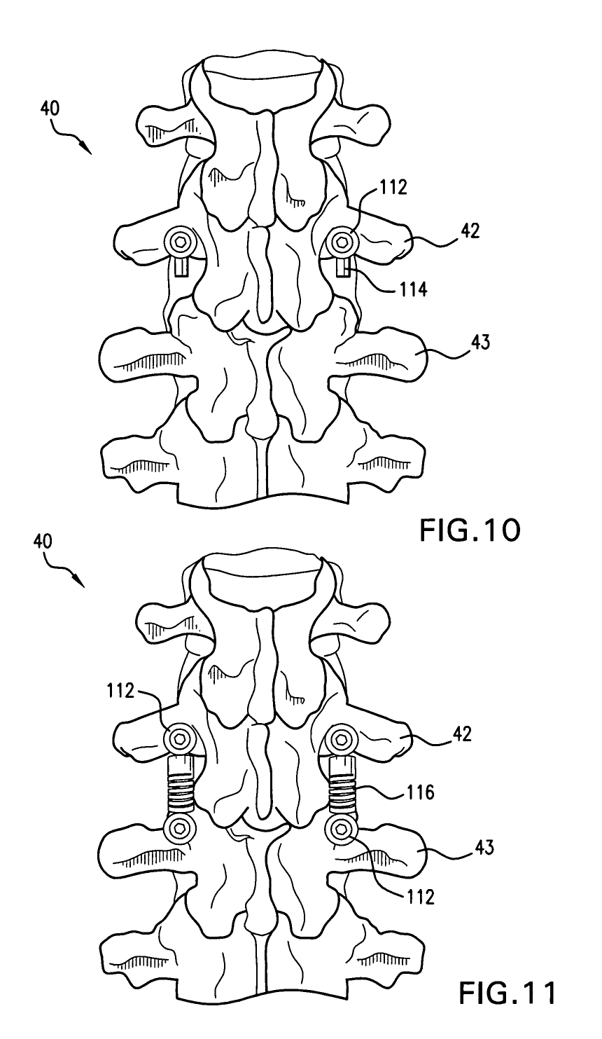

FIGS. 10 and 11 show steps that may be taken when installing a spinal support system in accord with the present invention. FIGS. 10 and 11 show a spine 40 having four vertebrae, 41 through 44. In both figures the pedicle screws are shown having already been installed. In FIG. 10, which precedes the steps shown in FIG. 11, inserts 14 have also been installed into the pedicle screws. As can be seen in FIG. 10, these inserts protrude out from the pedicle screws and have been installed in the top pedicle screw. FIG. 11 shows a later step after the bridging springs have been installed. As can be seen, the springs slide up and over the insert 114 and also slide into the lower pedicle screws. As discussed above, the coiled portion of the pedicle springs 1 16 in this example is positioned towards the lower vertebrae 43, while the solid portion of the pedicle spring 116 is positioned towards the upper vertebrae 42. Positioning the spring in this fashion can more closely mimic the strength features of a facet joint.

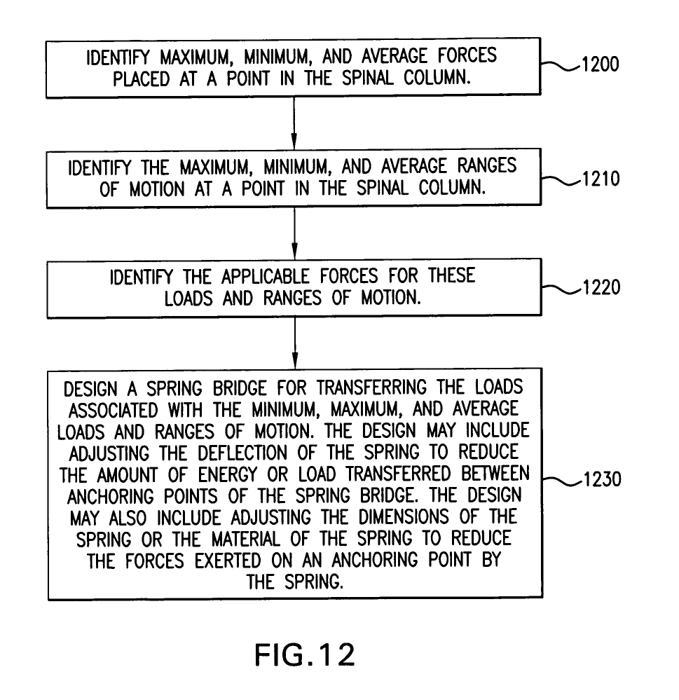

FIG. 12 is a flow chart depicting methods that may be used in accord with the present invention. These methods may include steps taken to evaluate loads placed on the spine along each of three orthogonal axes and to design a spinal support system for transferring some or all of these forces between two points. This method may include identifying the minimum, maximum, and average loads placed on a point or several points of the spine. These forces may be determined for an individual as well as for a typical patient and for a class of patients. These classes of patients can include classes based on sex, weight ranges and height ranges. The method may also include identifying the minimum, maximum, and average ranges of motion for one or several points on the spine of an average patient well as for a specific individual. The applicable loads to generate these forces and ranges of motion may then be determined. This may include generating loads in each of the applicable three axes. Using these loads or the underlying data, a bridging spring may be designed to transfer some or all of these loads between two points in the spine. This spring may be designed to absorb energy as the load is transferred between points and may also be designed not to carry loads in certain planes of movement. The spring may be designed to reduce the load placed on an anchoring point in order to increase the longevity or the cycle length of an anchor installed in the spine.

While various embodiments are discussed throughout and shown in the drawings, other embodiments are also possible. Features from one embodiment may be mixed with features from another. Features may also be deleted or added while remaining within the scope of the invention. Likewise, the methods described herein reflect embodiments that, too, may be modified without departing from the present invention.

What is claimed is:

1. A spinal support system comprising:
a first spinal anchor and a second spinal anchor,
a bridge coupled to the first spinal anchor and the second spinal anchor,
the bridge having a length, the length including a more flexible section and a less-flexible section
wherein the less-flexible section is at an end of the bridge and the more flexible section is off-centered between the first spinal anchor and the second spinal anchor.

2. The system of claim 1 wherein the first spinal anchor is a pedicle screw and the second spinal anchor is a pedicle screw.

3. The system of claim 1 wherein the bridge is a spring having coils with a rectangular cross-section.

4. The system of claim 4 wherein the more flexible section is a coiled section and the less-flexible section is a non-coiled section.

5. The system of claim 1 wherein the less-flexible section comprises one third of the distance between the anchors and the more flexible section comprises two-thirds of the distance between the anchors.

6. The system of claim 1 wherein the less-flexible section is positioned above the more flexible section when the bridge is coupled to the first anchor and the second anchor.

7. The system of claim 1 further comprising a spacer, the spacer positioned between the bridge and the anchor, the spacer fitting within the anchor, the spacer decoupleable from the bridge.

8. The system of claim 7 wherein the spacer is a tubular and covers a portion of the bridge.

9. A spinal stabilization kit comprising:
a plurality of the spinal stabilization systems of claim 1.

10. The kit of claim 9 further comprising:
a plurality of spacers, the spacers sized to couple with one or more of the bridges in the kit.

11. The kit of claim 10 wherein the bridges are springs having a coiled section and a non-coiled section and wherein the anchors are pedicle screws.

12. The kit of claim 9 wherein the spacers are tubular in shape and are configured with a bore that is sized to receive an end of a spring bridge.

13. The kit of claim 9 wherein one or more of the bridges comprises titanium.

14. The kit of claim 9 wherein one or more of the bridges comprises a shape memory alloy.

15. A method of designing a spinal stabilization system comprising:
identifying three-dimensional loads placed at a location of a spinal column, the loads identified along orthogonal axes, an x-axis, a y-axis, and a z-axis;
identifying three-dimensional ranges of motion of that location of the spinal column for each of the x-axis, the y-axis, and the z-axis;
quantifying the forces associated with the identified loads and the identified ranges of motion along each three dimensional axis, the x-axis, the y-axis, and the z-axis; and
designing a spring bridge to transfer some but not all of the loads for at least one of the axes from one end of the spring bridge to another end of the spring bridge, the load not transferred being absorbed at least partially by flexure of the spring bridge.
